(19) United States
(12) Patent Application Publication
(10) Pub. No.: US 2019/0256491 A1
(43) Pub. Date: Aug. 22, 2019

(54) Methods For Treatment Of 16P11.2 Microdeletion Syndrome

(71) Applicant: Massachusetts Institute of Technology, Cambridge, MA (US)

(72) Inventors: Mark F. Bear, Cambridge, MA (US); Di Tian, Cambridge, MA (US)

(21) Appl. No.: 16/279,441

(22) Filed: Feb. 19, 2019

Related U.S. Application Data
(63) Continuation of application No. 14/521,707, filed on Oct. 23, 2014.
Application No. 14/521,707 is a continuation of application No. PCT/US2013/038179, filed on Apr. 25, 2013.
(60) Provisional application No. 61/638,616, filed on Apr. 26, 2012.

(51) Int. Cl. C07D 401/06; A61K 31/4439; A61K 31/198; A61K 31/382

(52) U.S. Cl. CPC C07D 401/06; A61K 31/382; A61K 31/198; A61K 31/4439

(57) ABSTRACT

Subjects that have a 16p11.2 microdeletion syndrome are treated by administering compositions that include mGluR inhibitors, including mGluR antagonists that include mGluR negative allosteric modulators. Administration of compositions employed in the methods of the invention can treat psychiatric, including neuropsychiatric disorders, cognitive impairments, attention, obesity, intellectual disability and seizure disorders.

### RELATED APPLICATIONS

This application is a continuation of U.S. application Ser. No. 14/521,707, filed on Oct. 23, 2014, which itself is a continuation of International Application No. PCT/US2013/038179, which designated the United States and was filed on Apr. 25, 2013, published in English. International Application No. PCT/US2013/038179 claims the benefit of U.S. Provisional Application No. 61/638,616, filed on Apr. 26, 2012. The entire teachings of the above applications are incorporated herein by reference.

### GOVERNMENT SUPPORT

This invention was made with government support under Grant No. R21 MH090452 awarded by The National Institutes of Health. The government has certain rights in the invention.

### BACKGROUND OF THE INVENTION

16p11.2 microdeletion syndrome is caused by a deletion of about 600 kilobases near the middle of chromosome 16 at position p11.2. The deletion affects one of two copies of chromosome 16 in each cell. The 600 kb region may contain at least about 25 genes, the function of which many remain unknown. Humans with 16p11.2 microdeletion syndrome generally have developmental delays, intellectual disabilities and delays in speech and language skills. In addition, some features of autism spectrum disorder have been reported in humans with 16p11.2 deletion disorder. In humans with 16p11.2 microdeletion syndrome, expressive language skills (vocabulary and the production of speech) are generally more severely affected than receptive language skills. Currently, treatment for humans with 16p11.2 deletion disorder include use of drugs to control problem behaviors, including antipsychotic, and physical and psychological therapies. Thus, there is a need to develop new and improved methods of treating a subject with a 16p11.2 microdeletion syndrome.

### SUMMARY OF THE INVENTION

The present invention is related to methods of treating a 16p11.2 microdeletion syndrome in a subject.

In an embodiment, the invention is a method of treating a psychiatric disorder in a subject having a 16p11.2 microdeletion syndrome, comprising the step of administering a composition that includes a Group I mGluR inhibitor.

In another embodiment, the invention is a method of treating a subject with a 16p11.2 microdeletion syndrome by administering a composition that includes Formula I.

In yet another embodiment, the invention is a method of treating a psychiatric disorder in a subject having a 16p11.2 microdeletion syndrome, comprising the step of administering a composition that includes a Group I mGluR antagonist, including a negative allosteric modulator of Group I mGluR.

The methods of the invention can be employed to treat subjects with 16p11.2 microdeletion syndrome, in particular, psychiatric and related behavioral disorders in the subject. Advantages of the claimed invention include, for example, safe and effective methods to treat of conditions associated with 16p11.2 microdeletion syndrome that have the potential to normalize central nervous system function consequent to the 16p11.2 microdeletion syndrome and thereby significantly improve the quality of life of humans with 16p11.2 microdeletion syndrome.

### BRIEF DESCRIPTION OF THE DRAWINGS

FIGS. 1A and 1B demonstrate that basal synaptic transmission is not altered in chr7qF3 mutant mice.

FIGS. 2A-2F demonstrate that Chr7qF3 mutant mice exhibit mGluR-LTD that is protein synthesis independent.

FIGS. 3A-3C demonstrated that Chr7qF3 mutant mice have deficits in hippocampal-dependent contextual fear conditioning and inhibitory avoidance.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The invention is generally directed to methods of treating subjects having a 16p11.2 microdeletion syndrome.

In an embodiment, the invention is method of treating a psychiatric disorder in a subject having a 16p11.2 microdeletion, comprising the step of administering a composition that includes a Group I mGluR inhibitor.

Psychiatric disorders that can be treated by methods of the invention include schizophrenia. The psychiatric disorders treated by the methods of the invention can be a neuropsychiatric disorder, such as at least one member selected from the group consisting of anxiety and attention deficit hyperactivity disorder.

Well-established methods to diagnosis subjects with a 16p11.2 microdeletion, including subjects that have a 16p11.2 microdeletion syndrome that have psychiatric and neuropsychiatric disorders, are known to one of ordinary skill in the art. For example, 16p11.2 microdeletions can be detected by clinical oligonucleotide array genomic hybridization (aGH) platforms, bacterial artificial chromosome (BAC)-based platforms, multiplex ligation-dependent probe amplification (MLPA), metaphase fluorescence in situ hybridization (FISH), and quantitative polymerase chain reaction PCR (qPCR) (Pagon, R. A., et al., GeneReviews, National Library of Medicine, Seattle, Wash., University of Seattle, Seattle, Wash.).

Routine, well-established clinical criteria and techniques can be be employed to identify subjects treated by the methods of the invention that have a psychiatric disorder, such as schizophrenia, and neuropsychiatric disorders, such as anxiety and attention deficit hyperactivity disorder (see, for example, Diagnostic and Statistical Manual of Mental Disorders, Fourth Edition (DSM-IV)).

mGluRs are a heterogeneous family of glutamate G-protein coupled receptors. mGluRs are classified into three groups. Group I receptors (mGluR1 and mGluR5) can be coupled to stimulation of phospholipase C resulting in phosphoinositide hydrolysis and elevation of intracellular calcium levels, modulation of ion channels (e.g., potassium channels, calcium channels, non-selective cation channels) and N-methyl-D-aspartate (NMDA) receptors. mGluR5 can be present on a postsynaptic neuron. mGluR1 can be present on a presynaptic neuron and/or a postsynaptic neuron. Group II receptors (mGluR2 and mGluR3) and Group III receptors (mGluRs 4, 6, 7, and 8) inhibit cAMP formation and G-protein-activated inward rectifying potassium channels. Group II mGluRs and Group III mGluRs are negatively coupled to adenylyl cyclase, generally present on presynaptic neurons, but can be present on postsynaptic neurons and function as presynaptic autoreceptors to reduce glutamate release from presynaptic neurons.

The methods of the invention can be employed in Group I mGluR inhibitors that are Group I mGluR antagonists (mGluR1 antagonist, mGluR5 antagonist). Group I mGluR antagonists include Group I mGluR negative allosteric modulators. Group I mGluR inhibitors can be employed in the methods of the invention alone or in combination with other mGluR inhibitors, such as Group III mGluR inhibitors, in particular mGluR7 antagonists, which can include mGluR7 negative allosteric modulators.

The Group I mGluR inhibitors administered to the subject can be an mGluR1 negative allosteric modulator, an mGluR5 negative allosteric modulator, or a combination of an mGluR1 negative allosteric modulator and an mGluR5 negative allosteric modulator. In a preferred embodiment, the negative allosteric modulator employed in the methods of the invention would achieve about 50%, about 60%, about 70%, about 80%, about 86%, about 90%, about 95% and about 100% occupancy of mGluR. Techniques to assess mGluR occupancy are well know and established cell and molecular biological techniques (see, for example, Lindemann, L., et al., J. Pharmacology and Experimental Therapeutics 339:474-486 (2011)).

Allosteric modulators are substances that indirectly modulate the effects of an agonist or inverse agonist at a target protein, for example a receptor. Allosteric modulators bind to a site distinct from that of the orthosteric agonist binding site. Generally, allosteric modulators induce a conformational change in protein structure, such as a receptor, including a mGluR. A positive allosteric modulator (PAM) induces an amplification, a negative modulator (NAM) attenuates the effects of the orthosteric ligand without triggering a functional activity on its own in the absence of the orthosteric ligand.

Negative allosteric modulators (NAM) employed in the methods of the invention attenuate a neuronal response to glutamate. Negative allosteric modulators employed in the methods of the invention can bind to an allosteric site on the mGluR complex and negatively affect neuronal signaling and subsequent intracellular signaling to thereby decrease mGluR-mediated neuronal signaling by, for example, decreasing G-protein coupled receptor signal transduction. NAMs employed in the methods of the invention may not affect binding of glutamate to the mGluR.

In an embodiment, the mGluR5 negative allosteric modulator (NAM) for use in the methods of the invention is a mGluR5 NAM that has inverse agonist properties, such as 2-chloro-4-((2,5-dimethyl-1-(4-(trifluoromethoxy)phenyl (-1H-imidazol-4-yl(ethyny)pyridine (CTEP) of Formula I (Lindemann, L., et al., J. Pharmacology and Experimental Therapeutics 339:474-486 (2011)) depicted below:

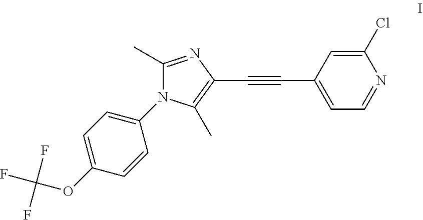 [SMILES: Cc1nc(C#Cc2ccnc(Cl)c2)c(C)n1-c1ccc(OC(F)(F)F)cc1]

Other exemplary mGluR negative allosteric modulators for use in the invention include Formula II (MPEP, 2-methyl-6-(phenylethynyl)-pyridine), Formula III (MTEP 3-[(2-methyl-1,3-thiazol-4-yl)ethynyl]pyridine) and Formula IV (Fenobam, [N-(3-chlorophenyl)-N-(4,5-dihydro-1-methyl-4-oxo-1H-imidazole-2-yl)urea]) depicted below:

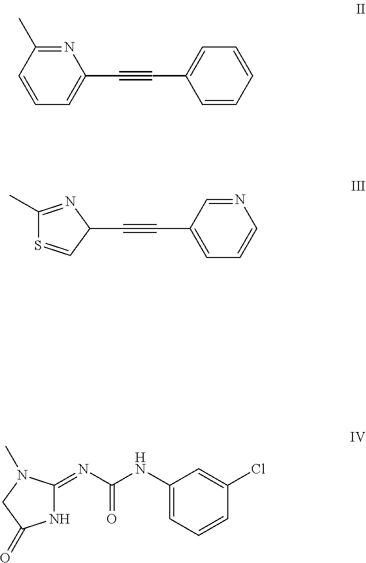

In an embodiment, the subject treated by the methods of the invention is a human subject. The human subject that has a 16p11.2 microdeletion syndrome and can further have autism spectrum disorder. Autism spectrum disorder is a group of pervasive developmental disorders. Autism spectrum disorder can be diagnosed employing established criteria well known to one of ordinary skill in the art (see, for example, Heurta, M. Pediatr. Clin. North Am. 590:103-11 (2012) and Diagnostic and Statistical Manual of Mental Disorders, Fourth Edition (DSM-IV)). Criteria for consideration in a diagnosis of autism spectrum disorder include impairments in social interaction, impairments in communication and restricted, repetitive, and stereotyped patterns of behavior, interests and activities. Considerations in impairments in social interaction include marked impairment in the use of multiple nonverbal behaviors, such as eye-to-eye gaze, facial expression, body postures, and gestures to regulate social interaction; failure to develop peer relationships appropriate to developmental level; a lack of spontaneous seeking to share enjoyment, interests, or achievements with other people; and lack of social or emotional reciprocity. Considerations for impairments in communication can include a delay in, or total lack of, the development of spoken language; marked impairment in the ability to initiate or sustain a conversation with others; stereotyped and repetitive use of language or idiosyncratic language; lack of varied, spontaneous make-believe play or social imitative play appropriate to developmental level; and restricted, repetitive, and stereotyped patterns of behavior, interests, and activities.

In yet another embodiment, the invention is a method of treating a subject having 16p11.2 microdeletion syndrome by administering a mGluR antagonist. The mGluR antagonist can be administered alone or in combination with the mGluR NAM to the subject. In a particular embodiment, the subject is administered a Group I mGluR antagonist (mGluR1 antagonist, mGluR5 antagonist). Group I mGluR antagonists can be employed in the methods of the invention in combination with a mGluR7 antagonist.

Antagonists can act at the level of the ligand-receptor interactions, such as by competitively or non-competitively (e.g., allosterically) inhibiting ligand binding. The antagonist can act downstream of the receptor, such as by inhibiting receptor interaction with a G protein or downstream events associated with G protein activation, such as stimulation of phospholipase C or extracellular signal regulated kinase (ERK), elevation in intracellular calcium, the production of or levels of cAMP or adenylcyclase, stimulation and/or modulation of ion channels (e.g., K+, Ca++) (see, for example, Zhang, L., et al., J. Pharma Col. Exp. Ther. 300:149-156 (2002)). Exemplary mGluR antagonists for use in the methods of the invention include Formulas V-VII depicted below:

### (+)-2-amino-2-(3-trans-carboxycyclobutyl-3-(9-thioxanthyl)propionic acid)

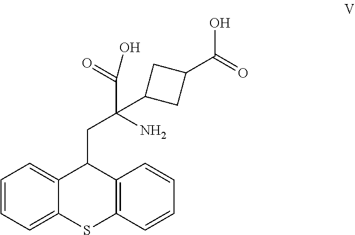 [SMILES: NC(CC1c2ccccc2Sc2ccccc21)(C(=O)O)C1CC(C(=O)O)C1]

### (S)-(+)-alpha-amino-4-carboxy-2-methylbenzeneacetic acid

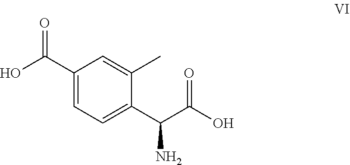

### (R,S)-1-aminoindan-1,5-dicarboxylic acid

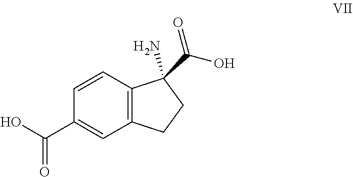 [SMILES: N[C@]1(C(=O)O)CCc2cc(C(=O)O)ccc21]

Subjects administered mGluR NAMs (e.g., Group I mGluR NAMs), mGluR antagonists of the invention, alone in in combination with mGluR NAMs, can have a psychiatric disorder (e.g., schizophrenia), a neuropsychiatric disorder (e.g., anxiety, attention deficit hyperactivity disorder), can be obese, have an intellectual disability and seizures.

In another embodiment, the invention is a method of treating a psychiatric disorder in a subject having a 16p11.2 microdeletion syndrome, comprising the step of administering a composition that includes a Group I mGluR antagonist.

In a further embodiment, the invention is a method of treating a subject having a 16p11.2 microdeletion syndrome, comprising the step of administering a composition that includes a Group I mGluR negative allosteric modulator.

The subject treated by the methods of the invention can have an improvement in a cognitive impairment consequent to administration of the compositions employed in the methods of the invention. The improvement in the cognitive impairment is an improvement in at least one member selected from the group consisting of memory (short term memory, long term memory, working memory, declarative memory) attention, executive function.

An “effective amount,” also referred to herein as a “therapeutically effective amount,” when referring to the amount of a compound (e.g., Formula I) or composition (e.g., pharmaceutical composition containing Formula I) that treats the subject having a 16p11.2 microdeletion syndrome (e.g., treating a psychiatric disorder), is defined as that amount, or dose, of a compound or composition that, when administered to a subject is sufficient for therapeutic efficacy (e.g., an amount sufficient to reduce clinical indicia of a psychiatric disorder, autism spectrum disorder, anxiety, attention deficit hyperactivity disorder, obesity, seizure disorder, intellectual disability or improve attention and cognition in the subject).

The methods of the present invention can be accomplished, for example, by the administration of a composition by enteral or parenteral means. Specifically, the route of administration is by oral ingestion (e.g., tablet, capsule form). Other routes of administration as also encompassed by the present invention including intramuscular, intravenous, intraarterial, intraperitoneal, or subcutaneous routes and nasal administration. Suppositories or transdermal patches can also be employed.

Compositions that include Group I mGluR inhibitors, mGluR NAMs and mGluR antagonists can be co-administered. Coadminstration can include simultaneous or sequential administration of the compositions that include Group I mGluR inhibitors, mGluR NAMs and mGluR antagonists.

Compositions employed in the methods of the invention can be administered alone or as admixtures with conventional excipients, for example, pharmaceutically, or physiologically, acceptable organic, or inorganic carrier substances suitable for enteral or parenteral application which do not deleteriously react with the compounds. Suitable pharmaceutically acceptable carriers include water and salt solutions, such as Ringer's solution, which do not deleteriously react with the compositions of employed in the methods of the invention. The preparations can also be combined, when desired, with other active substances to reduce metabolic degradation. The compositions that include Group I mGluR inhibitors (e.g., mGluR NAMs and mGluR antagonists) can be administered in a single or multiples doses (e.g., 2, 3, 4, 5, 6, 7, 8, 9, 10) over a period of time to confer the desired effect to treat the subject having a 16p11.2 microdeletion syndrome.

The compositions employed in the methods of the invention can be include Group I mGluR inhibitors administered in a dose of between about 0.1 mg/kg to about 1 mg/kg body weight; about 1 mg/kg to about 5 mg/kg body weight; between about 5 mg/kg to about 15 mg/kg body weight; between about 10 mg/kg to about 25 mg/kg body weight; between about 25 mg/kg to about 50 mg/kg body weight; or between about 50 mg/kg body weight to about 100 mg/kg body weight. The compounds can be administered in doses of about 0.01 mg, about 0.1 mg, about 1 mg, about 2 mg, about 10 mg, about 25 mg, about 50 mg, 100 mg, about 200 mg, about 250 mg, about 300 mg, about 350 mg, about 400 mg, about 500 mg, about 600 mg, about 700 mg, about 900 mg, about 1000 mg, about 1200 mg, about 1400 mg, about 1600 mg or about 2000 mg.

The dosage and frequency (single or multiple doses) administered to a subject can vary depending upon a variety of factors, including the severity of the psychiatric disorder, whether the subject suffers from other disorders, conditions or syndromes, kind of concurrent treatment (e.g., antipsychotic medications), or other health-related problems. Other therapeutic regimens or agents can be used in conjunction with the methods of the present invention. Adjustment and manipulation of established dosages (e.g., frequency and duration) are well within the ability of those skilled in the art. 0050.2200-003 (MIT-15581)

### EXEMPLIFICATION

Autism Spectrum Disorder (ASD) has a complex genetic landscape. Many genes and genetic loci have been linked to autism ((Geschwind, 2009) (Abrahams and Geschwind, 2010)). Among several types of autism-associated genetic abnormalities, chromosome copy number variation (CNV) is present in about 10-20% of ASD patients. Human chromosome 16p11.2 microdeletion syndrome is a common CNV in ASD and account for about 1% of cases ((Christian et al., 2008; Kumar et al., 2008; Weiss et al., 2008)). A mouse model of human chr16p11.2 microdeletion syndrome has shown phenotypes recapitulating some behavioral abnormalities and co-morbidities associated with autism ((Horev et al., 2011)). However, the pathophysiological and biochemical mechanisms underlying these behavioral phenotypes remain unknown. Little is known about how CNVs, as a distinct group of genetic abnormalities, contribute to autism spectrum disorder nor is there a single underlying neuropathophysiology linked to ASD associated with CNV. Elucidating the pathophysiology of CNV-associated autism will increase the understanding of the disease and help to develop effective therapeutic interventions.

Single-gene disorders have been associated with an increased rate of ASD that affect proteins known to modulate synaptic mRNA translation, such as FMRP in fragile X syndrome (FX), TSC1/2 in Tuberous Sclerosis Complex (TSC), and PTEN in Cowden syndrome (PTEN hamartoma syndrome). However, mouse models of FX and TSC (Fmr1−/γ (KO) and Tsc2+/−) mice show that there is no unified core pathophysiology underlying ASD. For example, although there is altered basal protein synthesis in both FX and TSC model mice, and rectification of this defect by genetic and/or pharmacological approaches results in an amelioration of impairments in synaptic plasticity and correction of behavioral abnormalities, the approaches to treat impairments are polar opposites ((Auerbach et al., 2011; Dolen et al., 2007)).

Several of the deleted genes in the human chr16p11.2 region play important roles in the MAPK and mTor signaling pathways (Table 1), which have been implicated in altered (and polar opposite) protein synthesis regulation in Fmrl KO mice ((Osterweil et al., 2010; Sharma et al., 2010)). The data described herein show that a mouse model for human chromosome 16p11.2 microdeletion syndrome share some, not all, of the aspects of pathophysiology with the Fmrl knockout mouse.

TABLE 1
List of genes at human chromosome 16p11.2 syndrome that have putative or
known CNS functions.

| | General | | |
|---|---|---|---|
| Genes | Functions | CNS Functions | References |
| Coronin1a | Actin binding | Unknown; but a family member, | Hasse et al., 2005 |
| (Coronin-like protein | protein | Coronin 3, is involved in brain | Mueller et al., 2008 |
| A) | T-cell trafficking | morphogenesis | |
| MAPK3 | MAP kinase | MAPK pathway is involved in | Selcher et al., 2001 |
| (Erk1) | | plasticity; Erk1 KO mice show mild | Sweatt, 2004 |
| | | learning deficits | |
| Ppp4c | Serine/threonine | Activate mTOR and NF-k B pathway; | Cohen et al., 2005 |
| (Protein phosphotase | phosphotase 4 | interact c/survival motor neuron | |
| 4c) | catalytic subunit | complex | |
| DOC2α | Ca⁺⁺-binding protein | Synaptic vesicle associated Ca⁺⁺- | Sakaguchi et al., |
| (C2 domain protein) | | binding protein; regulating vesicle | 1999 |
| | | release; KO mice show impaired LTP | Groffen et al., 2006 |
| | | and passive avoidance task | Verhage et al., 1997 |
| Taok2 | Serine/threonine | Activity-induced N-cadherin | Huangfu et al., 2006 |
| (Thousand and one | kinase; | endocytosis | |
| kinase 2) | activate p38 and JNK | | |
| | MAP kinase pathway | | |
| Sez6L2 | Transmembrane | Unknown; but Sez6 KO mice show | Gunnersen et al., |
| (Seizure 6 like protein | protein | excessive short dendrites and | 2007 |
| 2) | | neuritic branching | |
| Cdipt | Catalyze | Unknown; but may involved in PI3K | Saito et al., 1998 |
| (Phosphatidylinositol | biosynthesis of | signaling pathway | Nielsen, 2008 |
| synthase) | phosphatidylinotiol | | |
| MVP | Structural protein in | Unknown; maybe involved in multi- | Kolli et al., 2004 |
| (Major vault protein) | ribonucleo-protein | drug resistance in brain tumors; | Steiner et al., 2006 |
| | particles--vaults; | expressed in nucleus-neurite axis; | Kim et al., 2006 |
| | associated c/ | maybe involved in mRNA transport | Paspalas et al., 2008 |
| | microtubules; | | |
| | activate PI3K and | | |
| | MAPK pathway | | |

As described herein, a mouse model of human chr16p11.2 microdeletion syndrome showed selective differences in metabotropic glutamate receptor (mGluR) mediated synaptic plasticity and hippocampus-associated behaviors. Specifically, (1) the heterozygous mutant mice had normal basal synaptic transmission as revealed by assays of input-output functions and paired pulse facilitation; (2) these mice have normal NMDA-receptor mediated synaptic potentiation and depression; (3) unlike wild-type animals, mGluR-mediated long-term depression is independent of protein synthesis in mutant mice; (4) mutant mice exhibit significant cognitive impairments in contextual fear conditioning and inhibitory avoidance extinction (IAE) tests; and (5) chronic treatment with CTEP, an mGluR5 antagonist (specifically an mGluR5 negative allosteric modulator), significantly ameliorates the cognitive impairment in young adult mutant mice in an inhibitory avoidance extinction test.

### Materials and Methods

Animals.

A mouse line carrying a heterozygous microdeletion of chr7qF3, the syntenic region of human chr16p11.2 was used in this study ((Horev et al., 2011)). These mice were backcrossed to C57BL/6J mice from Charles River Laboratory for a minimum of five generations. Genotyping was performed by PCR analyses. Mice were group housed on a 12 hour on/12 hour off light, dark cycle.

Reagents.

S-3,5-dihydrozyphenylglycine (S-DHPG) was purchased from Sigma-Aldrich. Fresh aliquots of DHPG was prepared in H₂O as 100× stock and used within 7 days of preparation. Cycloheximide (CHX) was purchased from Sigma-Aldrich, prepared fresh in H₂O as 100× stock and used on the same day of experimentation. CTEP, [2-chloro-4-((2,5-dimethyl-1-(4-(trifluoromethoxy)phenyl)-1H-imidazol-4-yl)ethynyl)pyridine] (Formula I), was employed in these experiments.

Hippocampal Electrophysiology.

Electrophysiological experiments were performed at the Schaffer collateral-CA1 synapse of hippocampal slices prepared from p28 to p35 male mice using experimental protocols as previously described ((Auerbach et al., 2011)). Dorsal hippocampal slices (400 μm thick) were used in all recordings. Input-output functions were determined by incrementally (10 μA to 100 μA) stimulating the Schaffer collaterals and recording the resulting fEPSP response. Paired-pulse facilitation was conducted by applying two stimulus pulses at varying inter-stimulus-intervals (ISI). Facilitation was measured by taking the ratio of the fEPSP slope in response to stimulus 2 to that of stimulus 1. For DHPG-LTD, slices were incubated in artificial cerebrospinal fluid (ACSF) in the presence or absence of the protein synthesis inhibitor cycloheximide (±CHX, 60 μM, 40 min), and mGluR5 was activated by bath application of DHPG (50 μM, 5 min). Synaptic responses were followed for an additional 60 min following DHPG application. For paired-pulse low frequency stimulation (PP-LFS) slices were incubated in ACSF containing APV (50 μM)±CHX for 30 min. mGluR5-LTD was then induced by application (20 min) of paired-pulse stimulation (50 ms ISI) at 1 Hz, and synaptic responses were recorded for an additional 60 min.

Contextual Fear Conditioning.

Contextual fear conditioning was performed as previously described ((Auerbach et al., 2011; Ehninger et al., 2008)). Briefly, 8 to 12 week-old WT and chr7qF3 mutant male mice were fear conditioned on day 1 and the subsequent percentage of time spent freezing in either the familiar or a novel context was determined about 24 hours later. On the day of conditioning, animals were allowed to explore the behavioral chamber for 3 min, followed by delivery of a single 0.8 mA (2s) foot shock. Mice remained in the context for about 15 sec after the shock, and then returned to their home cage. Conditioned fear response was tested about 24 hours later. To determine context specificity of the conditioned response, mice trained on day 1 were separated into two groups on day 2: one group was tested in the same training context (familiar context), the other tested in a novel context. The novel context was created by varying spatial cues, floor material, and lighting of the testing chamber. The percentage of time a mouse spent freezing during the test period (about 4 min session) was used as the behavioral readout. To determine if mutant mice had the same response to foot-shock as wildtype mice, the combined distance traveled during the about 2s foot-shock and about is immediately following were measured. Statistical significance was determined using two-way ANOVA and post hoc Student's t-tests.

Inhibitory Avoidance Extinction Test.

Inhibitory avoidance extinction (IAE) tests were performed as previously described with modification ((Dolen et al., 2007)). Briefly, 4-6 weeks male mice were divided into four groups according to genotype and CTEP treatment: WT+vehicle, WT+CTEP, Mutant+vehicle, and Mutant+CTEP. CTEP or vehicle was administered by oral gavage every other day for 4 weeks. The last dose was given about 16-20 hours prior to the training session. IA tests were conducted in a two-chambered Perspex box consisting of a lighted side and a dark side separated by a trap door. On the training day, mice were habituated in the behavioral room for about 2 hours before training. During training, a mouse was placed into the lit side of the chamber and allowed to explore for about 30 seconds before the trap door was opened. The ensuing time spent by the animal in the light chamber before entering the dark chamber was recorded as latency. Immediately after fully entering the dark side of the camber, subjects were given about a 2 sec mild foot shock (about 0.4 mA) and allowed to spend an additional 60 sec before being returned to their home-cage. The acquisition and expression of fear memory was tested at about 6, about 24, and about 48 hours post training. The testing protocol used was the same as the training protocol. Latencies to enter the dark side of the chamber were recorded and used as measurement of IAE performance. Statistical significance was determined using two-way ANOVA and post hoc Student's t-tests.

### Results

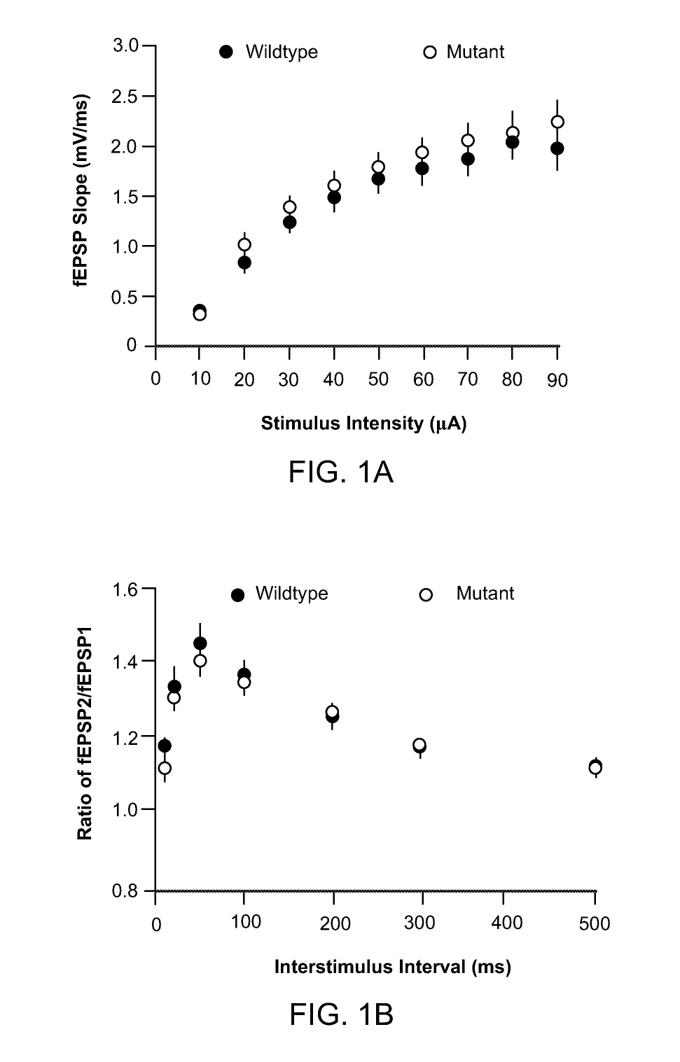

Basal synaptic transmission was analyzed in Schaeffer collateral-CA1 synapse by measuring input-output functions and paired pulse facilitation. Input-output functions do not differ between slices from WT and mutant mice (FIG. 1A). Similarly, paired-pulse facilitation in mutant slices was comparable to that observed in WT (FIG. 1B). In FIG. 1A, input-output functions, plotted as fEPSP slope versus stimulus intensity, do not differ between wildtype (n=14 animals) and chr7qF3 mutant mice (n=14 animals). In FIG. 1B, paired-pulse facilitation in chr7qF3 mutant (n=17 animals) mice is comparable to wildtype mice (n=16) across multiple stimulus intervals (10, 20, 50, 100, 200, 300, 500 ms). No statistically significant differences exist between wild type and chr7qF3 mutant mice at any stimulus intensity (FIG. 1A) or interstimulus interval (FIG. 1B) (Repeated measures ANOVA, p>0.5). All data are plotted as mean+SEM. This indicates that global synaptic function is normal in the mutant hippocampal slices.

Group I mGluR mediated synaptic plasticity was assessed. mGluR-LTD can be induced either by chemical induction by pharmacological stimulation of mGluRs (DHPG-LTD) or electrical induction by applying a series of paired pulses at about 50 ms interval (PP-LFS-LTD). Two independent expression mechanisms have been described in mGluR-LTD: reduced probability of presynaptic glutamate release and reduced post-synaptic expression of AMPA receptor ((Fitzjohn et al., 2001; Luscher and Huber, 2010; Mockett et al., 2011; Nosyreva and Huber, 2005)). In WT hippocampal slices, the post-synaptic component of mGluR-LTD requires rapid dendritic protein synthesis and can be blocked by a protein translation inhibitor, CHX ((Huber et al., 2000)). In contrast, mGluR-LTD in hippocampal slices from Fmrl KO and Tsc2^{+/-} mice are resistant to post-synaptic inhibition of protein synthesis ((Auerbach, et al., 2011; Nosyreva and Huber, 2005; Dolen et al., 2007; Huber et al., 2002; Michalon et al., 2012)). mGluR-LTD was assayed in the presence and absence of CHX.

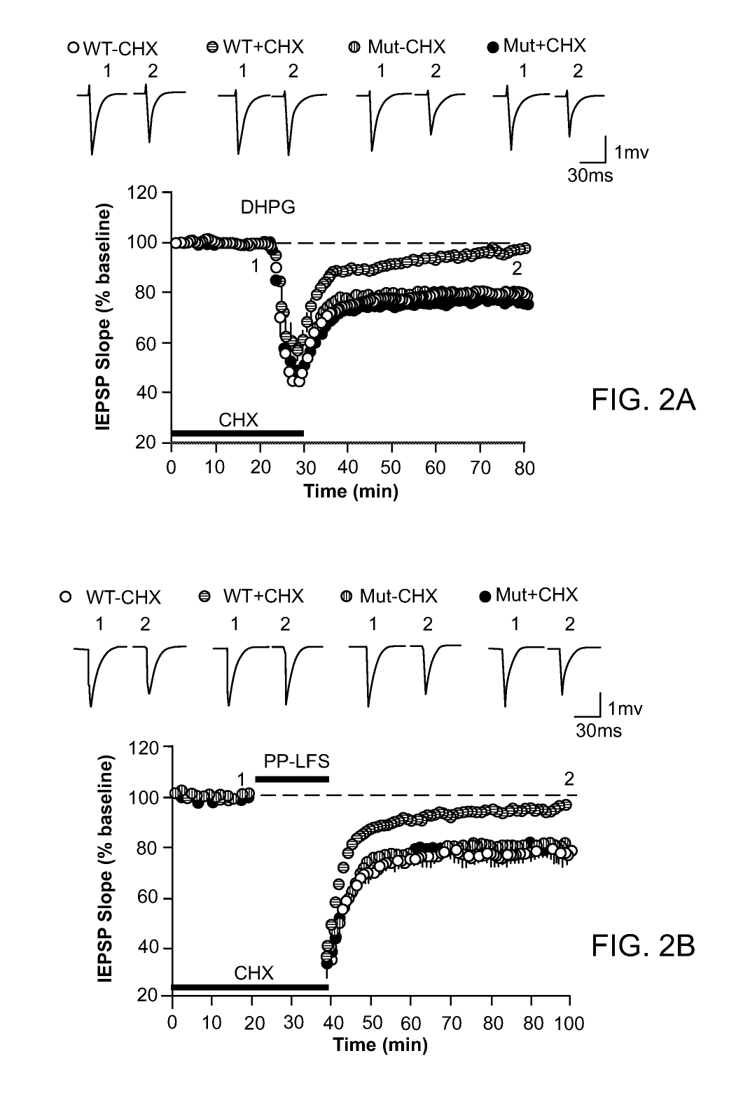

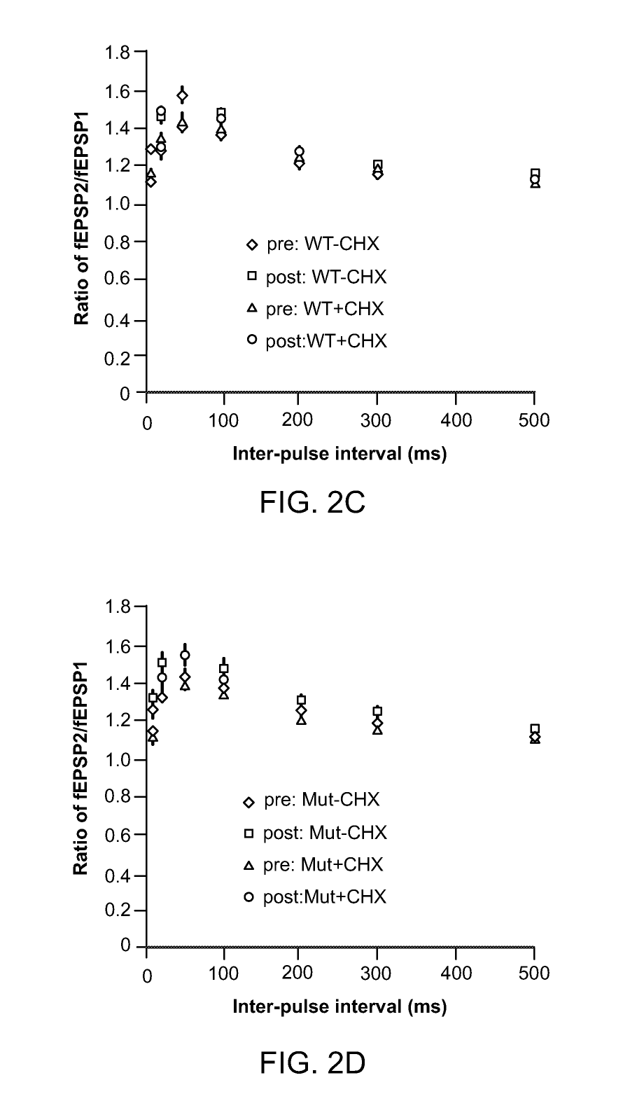

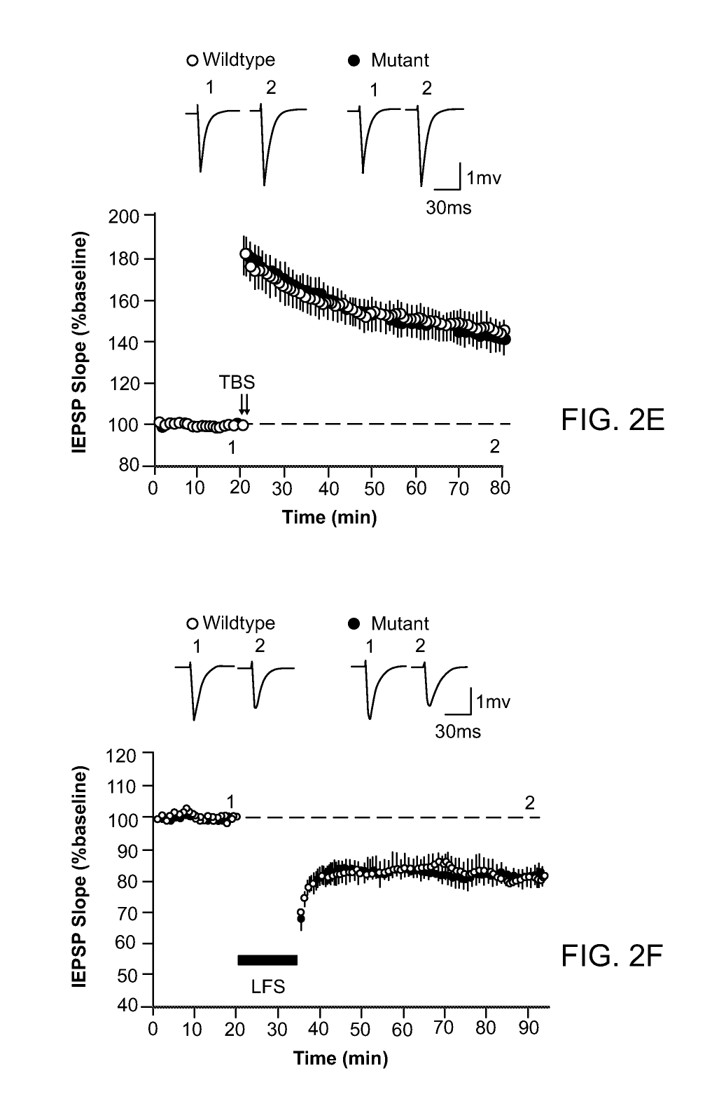

In the absence of CHX, the magnitude of depression in DHPG-LTD was comparable between WT and mutant slices (FIG. 2A). In WT slices, pre-treatment with CHX significantly blocked DHPG-LTD. However, the same treatment had no effect on mutant slices. To further confirm this observation, mGluR-LTD using the PP-LFS electrical induction protocol was assessed. The magnitude of depression is essentially the same between the WT and mutant slices in the absence of CHX. CHX almost completely blocked depression in WT and had no effect in mutant slices (FIG. 2B). This insensitivity of mGluR-LTD in chr7qF3 mutant mice to protein synthesis blockage resembles what has previously been described in Fmrl KO mice ((Dolen et al., 2007; Huber et al., 2002; Michalon et al., 2012)).

To test whether the difference in CHX sensitivity between WT and mutant slices was due to a different pre-synaptic response to either DHPG treatment or PP-LFS induction, paired pulse facilitation at the beginning and end of DHPG-LTD experiment (FIGS. 2C and 2D) was assessed. In both the WT (FIG. 2C) and mutant slices (FIG. 2D), DHPG treatment resulted in increased paired pulse facilitation, and hence reduced pre-synaptic glutamate release; and this was independent of CHX treatment. The magnitude of pre-synaptic weakening was comparable in WT and mutant regardless of CHX treatment (FIGS. 2C and 2D). These findings support the conclusion that a deficiency in post-synaptic regulation of protein synthesis is responsible for altered LTD in the chr7qF3 mutant mice.

Two types of NMDAR-mediated hippocampal plasticity were assessed to determine whether the deficits in mGluR-LTD observed in the mutant mice was due to a global disruption in synaptic plasticity. Theta-burst stimulation (TBS) induced long-term potentiation (TBS-LTP) that was indistinguishable between WT and mutant slices (FIG. 2E). Similarly, low-frequency stimulation induced long-term depression (LFS-LTD) was unaltered in mutant slices as compared to WT controls (FIG. 2F). These data demonstrate that other forms of hippocampal synaptic plasticity are unaltered in the mutant mice.

FIG. 2A shows the magnitude of DHPG-induced LTD is comparable in hippocampal slices from wildtype (WT, n=17 animals, 23 slices) and chr7qF3 mutant (Mut, n=17 animals, 26 slices) mice in the absence of the protein synthesis inhibitor cycloheximide (CHX). However, in the presence of CHX, DHPG-induced LTD is blocked in WT slices (WT, n=17 animals, 25 slices) while it remains unaffected in slices from chr7qF3 mutants (Mut, n=17 animals, 23 slices) (two-way ANOVA p<0.001). FIG. 2B shows the magnitude of PP-LFS LTD is comparable in hippocampal slices obtained from WT (n=12 animals, 17 slices) and chr7qF3 mutant (n=7 animals, 12 slices) mice in the absence of the protein synthesis inhibitor CHX. In contrast, in the presence of CHX, PP-LFS LTD is blocked in WT slices (n=12 animals, 15 slices), but it remains unaffected in slices from chr7qF3 mutant mice (n=7 animals, 11 slices) (two-way ANOVA p<0.001). FIGS. 2C and 2D show hippocampal paired-pulse facilitation is comparable in WT (n=17 animals, 18 slices for WT-CHX, 21 slices for WT+CHX) and chr7qF3 mutant (n=16 animals, 21 slices for Mut-CHX, 16 slices for Mut+CHX) mice. The data were recorded in the same animals and slices from which DHPG-LTD experiments were conducted (panel A). FIG. 2E shows LTP induced by application of theta-burst stimulation (TBS) is not altered in chr7qF3 mutants (n=9 animals, 17 slices) as compared to WT (10 animals, 19 slices) mice. FIG. 2F shows LTD induced by the application of low frequency stimulation (LFS) is not altered in chr7qF3 mutants (n=7 animals, 13 slices) as compared to WT (n=9 animals, 15 slices) mice. In FIGS. 2A, 2B, 2E and 2F, representative fEPSP traces (average of 10 sweeps) were taken at the times indicated by numerals.

The electrophysiological studies identified a specific deficit in mGluR-mediated synaptic plasticity. The mutant mice were the tested in two hippocampus-dependent behavioral assays: contextual fear conditioning and inhibitory avoidance extinction.

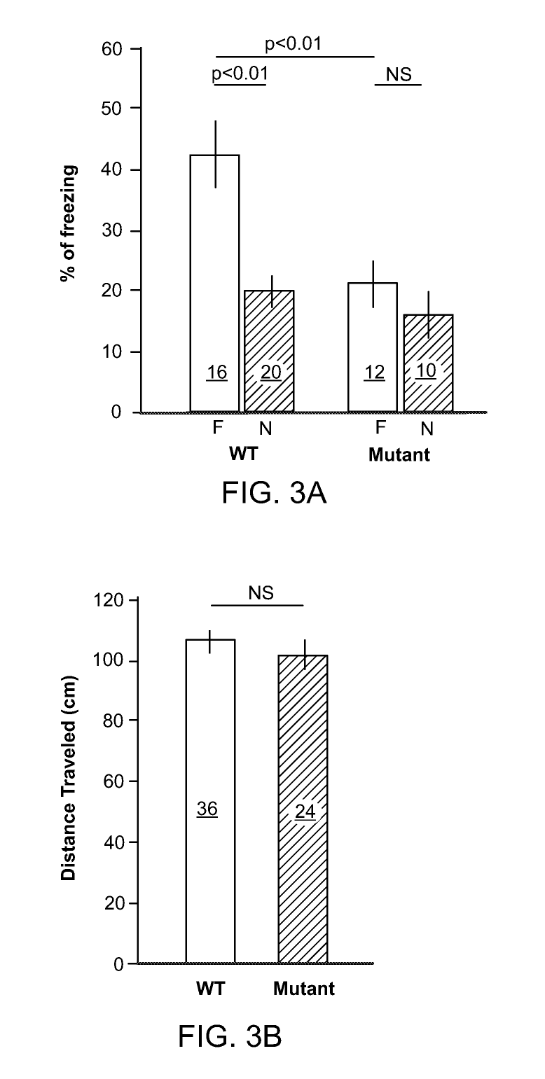

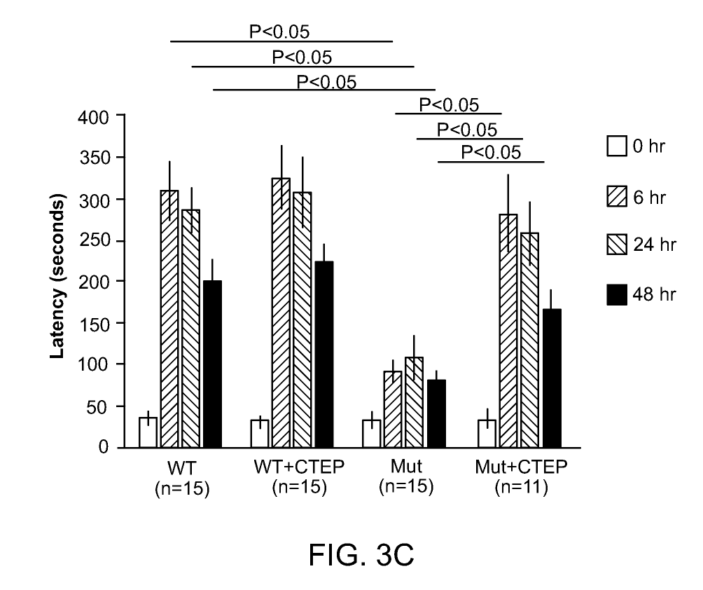

Contextual fear conditioning is a hippocampus-dependent one-trial learning paradigm. It requires intact mGluR5 signaling (Lu et al., 1997) and new protein synthesis at the time of conditioning. In this assay, mutant mice were exposed to a distinct environmental context, in which about a 2 sec foot-shock was delivered. Mice were expected to form a context-associated fear memory. Twenty-four hours after training, mice were exposed to either the same (familiar) or a different (novel) context. WT mice expressed the fear memory by freezing significantly more in the familiar than the novel context (FIG. 3A). FIG. 3A shows that Chr7qF3 mutant mice have deficits in discrimination between novel and familiar contexts. The Y-axis represents the percentage of time spent freezing during the 4 min testing period (performed 24 hours after initial foot shock). Numerals in each column represent the number of mice in each experimental group. (F, familiar context; N, novel context).

In contrast to WT, mutant mice showed significantly reduced freezing in the familiar context, and there was no distinction between the familiar and novel context. To determine if the difference in freezing time between mutant and WT mice was due to a difference in sensitivity to foot-shock, we measured the distance each animal traveled during the 2 sec foot-shock and 1 sec immediately following. As shown in FIG. 3B, the traveling distance was comparable between the two genotypes, indicating that the difference in freezing time in the familiar context between WT and mutant mice was likely due to a cognitive impairment in the latter group. FIG. 3B shows that Chr7qF3 mutant and wildtype mice show no difference in their motor response to foot shock during the initial training session. The Y-axis represents the average distance traveled during the 2 sec foot shock and 1 sec immediately following. No statistically significant difference in the distance traveled was found between genotypes (Student's t-test; p>0.05).

Mice were evaluated in another hippocampus-associated behavioral paradigm: inhibitory avoidance (IA). IA is a multi-phase test used to assay memory formation and extinction. During the training session (0 hr), mice were placed in the light chamber of a two-chamber box. After a variable latency in the light side, they entered the dark side of the box where about a 2 sec foot-shock was delivered. Acquisition and extinction of the fear memory, as measured by the latency to re-enter the dark chamber from the lighted side, was tested 6 hr (acquisition) as well as about 24 hr and about 48 hr (extinction) after training. Experiments were conducted to determine: (1) if chr7qF3 mice had deficits similar to Fmrl KO mice in the IA test, and (2) if deficits were present, could the mGluR5 inhibitor CTEP treatment ameliorate them. Mice were divided into four groups: WT+vehicle, WT+CTEP, Mut+vehicle, and Mut+CTEP. CTEP was given by the same dosing regime previously used with Fmrl KO mice ((Michalon et al., 2012)).

As shown in FIG. 3C, all four groups of mice had similar latency to enter the lit side of the chamber during the training session (0 hr). FIG. 3C shows that Chr7qF3 mutant mice show marked deficits in fear memory in an inhibitory avoidance task and these deficits are ameliorated by chronic CTEP treatment. As compared to WT mice, Chr7qF3 mutant mice show reduced latencies to re-enter the chamber where they received foot shock during test sessions at 6, 24, and 48 hours post training. Although having no effect on WT mice, CTEP treatment of Chr7qF3 mutant mice significantly lengthens their latency to re-enter the chamber at 6, 24, and 48 hours post training. Two-way ANOVA and post-hoc Student's t-test were used for statistical analyses. The WT+vehicle and WT+CTEP groups showed similar and significantly increased latencies to re-enter at 6 hr, indicating good acquisition of fear memory. Both groups also exhibited extinction at 48 hr. There was no statistically significant difference between these two groups at any time points (two-way ANOVA). In contrast, although the Mut+vehicle mice showed an increased latency to re-enter at 6 hr compared to 0 hr, the magnitude of this increase was significantly less than that observed in WT+vehicle mice. In addition, no extinction was observed in Mut+vehicle mice at either 24 hr or 48 hr. In marked contrast however, CTEP treatment dramatically lengthened the latency to re-enter the dark chamber at 6 hr in mutant mice (Mut+CTEP) to a level comparable to WT+vehicle mice. Moreover, mutant mice with CTEP treatment (Mut+CTEP) also showed significant extinction at 48 hr, similar to the two wildtype groups (WT+vehicle and WT+CTEP).

Interestingly, both the contextual fear conditioning and inhibitory avoidance revealed two similar cognitive deficits in chr7qF3 mice. First, mutant mice had impaired fear memory demonstrated by reduced freezing in CFC and shorter latency in IA. This is reminiscent of the memory deficit and intellectual disability seen in a high percentage of humans with autism. Second, the mutant mice lacked behavioral flexibility. This was demonstrated by the inability to distinguish the novel from familiar context in CFC and the lack of extinction in IA.

### Discussion

This study in a mouse model (chr7qF3) of human chr16p11.2 microdeletion syndrome focused on the hippocampus function, which is frequently impaired in children with autism. While basal synaptic transmission and NMDA-mediated plasticity were normal, mGluR5-mediated plasticity was altered in the mouse model. Specifically, mGluR5-LTD was no longer protein synthesis dependent in the mutant mice. Mutant mice had significant impairment in fear memory formation and reduced behavioral flexibility in two independent fear-conditioning paradigms. Moreover, cognitive deficits in IAE test were ameliorated by chronic oral administration of mGluR5 antagonist CTEP in the mutant mice.

These data show that mGluR5 mediated plasticity is compromised in the mouse model for human chr16p11.2 microdeletion. It is believed that abnormal mGluR5 function is responsible for cognitive impairment in the mutant mice since modulating mGluR5 can significantly improve the cognitive performance. Chr7qF3 mutant mice have no histological abnormality or major anatomical defects ((Horev et al., 2011)).

### REFERENCES

Abrahams, B. S., and Geschwind, D. H. (2010). Connecting genes to brain in the autism spectrum disorders. Archives of neurology 67, 395-399.
Auerbach, B. D., Osterweil, E. K., and Bear, M. F. (2011). Mutations causing syndromic autism define an axis of synaptic pathophysiology. Nature 480, 63-68.
Berry-Kravis, E. M., Hessl, D., Rathmell, B., Zarevics, P., Cherubini, M., Walton-Bowen, K., Mu, Y., Nguyen, D. V., Gonzalez-Heydrich, J., Wang, P. P., et al. (2012). Effects of STX209 (arbaclofen) on neurobehavioral function in children and adults with fragile X syndrome: a randomized, controlled, phase 2 trial. Science translational medicine 4, 152ra127.
Bozdagi, O., Sakurai, T., Dorr, N., Pilorge, M., Takahashi, N., and Buxbaum, J. D. (2012). Haploinsufficiency of Cyfip1 produces fragile X-like phenotypes in mice. PloS one 7, e42422.
Christian, S. L., Brune, C. W., Sudi, J., Kumar, R. A., Liu, S., Karamohamed, S., Badner, J. A., Matsui, S., Conroy, J., McQuaid, D., et al. (2008). Novel submicroscopic chromosomal abnormalities detected in autism spectrum disorder. Biological psychiatry 63, 1111-1117.
Dolen, G., Osterweil, E., Rao, B. S., Smith, G. B., Auerbach, B. D., Chattarji, S., and Bear, M. F. (2007). Correction of fragile X syndrome in mice. Neuron 56, 955-962.
Ehninger, D., Han, S., Shilyansky, C., Zhou, Y., Li, W., Kwiatkowski, D. J., Ramesh, V., and Silva, A. J. (2008). Reversal of learning deficits in a Tsc2+/− mouse model of tuberous sclerosis. Nature medicine 14, 843-848.
Fitzjohn, S. M., Palmer, M. J., May, J. E., Neeson, A., Morris, S. A., and Collingridge, G. L. (2001). A characterisation of long-term depression induced by metabotropic glutamate receptor activation in the rat hippocampus in vitro. The Journal of physiology 537, 421-430.
Geschwind, D. H. (2009). Advances in autism. Annual review of medicine 60, 367-380.
Horev, G., Ellegood, J., Lerch, J. P., Son, Y. E., Muthuswamy, L., Vogel, H., Krieger, A. M., Buja, A., Henkelman, R. M., Wigler, M., and Mills, A. A. (2011). Dosage-dependent phenotypes in models of 16p11.2 lesions found in autism. Proceedings of the National Academy of Sciences of the United States of America 108, 17076-17081.
Huber, K. M., Gallagher, S. M., Warren, S. T., and Bear, M. F. (2002). Altered synaptic plasticity in a mouse model of fragile X mental retardation. Proceedings of the National Academy of Sciences of the United States of America 99, 7746-7750.
Huber, K. M., Kayser, M. S., and Bear, M. F. (2000). Role for rapid dendritic protein synthesis in hippocampal mGluR-dependent long-term depression. Science 288, 1254-1257.
Kelleher, R. J., 3rd, and Bear, M. F. (2008). The autistic neuron: troubled translation? Cell 135, 401-406.
Kumar, R. A., KaraMohamed, S., Sudi, J., Conrad, D. F., Brune, C., Badner, J. A., Gilliam, T. C., Nowak, N.J., Cook, E. H., Jr., Dobyns, W. B., and Christian, S. L. (2008). Recurrent 16p11.2 microdeletions in autism. Human molecular genetics 17, 628-638.
Luscher, C., and Huber, K. M. (2010). Group 1 mGluR-dependent synaptic long-term depression: mechanisms and implications for circuitry and disease. Neuron 65, 445-459.
Michalon, A., Sidorov, M., Ballard, T. M., Ozmen, L., Spooren, W., Wettstein, J. G., Jaeschke, G., Bear, M. F., and Lindemann, L. (2012). Chronic pharmacological mGlu5 inhibition corrects fragile X in adult mice. Neuron 74, 49-56.
Mockett, B. G., Guevremont, D., Wutte, M., Hulme, S. R., Williams, J. M., and Abraham, W. C. (2011). Calcium/calmodulin-dependent protein kinase II mediates group I metabotropic glutamate receptor-dependent protein synthesis and long-term depression in rat hippocampus. The Journal of neuroscience: the official journal of the Society for Neuroscience 31, 7380-7391.
Nosyreva, E. D., and Huber, K. M. (2005). Developmental switch in synaptic mechanisms of hippocampal metabotropic glutamate receptor-dependent long-term depression. The Journal of neuroscience: the official journal of the Society for Neuroscience 25, 2992-3001.
Osterweil, E. K., Krueger, D. D., Reinhold, K., and Bear, M. F. (2010). Hypersensitivity to mGluR5 and ERK1/2 leads to excessive protein synthesis in the hippocampus of a mouse model of fragile X syndrome. The Journal of neuroscience: the official journal of the Society for Neuroscience 30, 15616-15627.
Sharma, A., Hoeffer, C. A., Takayasu, Y., Miyawaki, T., McBride, S. M., Klann, E., and Zukin, R. S. (2010). Dysregulation of mTOR signaling in fragile X syndrome. The Journal of neuroscience: the official journal of the Society for Neuroscience 30, 694-702.
Verpelli, C., Dvoretskova, E., Vicidomini, C., Rossi, F., Chiappalone, M., Schoen, M., Di Stefano, B., Mantegazza, R., Broccoli, V., Bockers, T. M., et al. (2011). Importance of Shank3 protein in regulating metabotropic glutamate receptor 5 (mGluR5) expression and signaling at synapses. The Journal of biological chemistry 286, 34839-34850.
Weiss, L. A., Shen, Y., Korn, J. M., Arking, D. E., Miller, D. T., Fossdal, R., Saemundsen, E., Stefansson, H., Ferreira, M. A., Green, T., et al. (2008). Association between microdeletion and microduplication at 16p11.2 and autism. The New England journal of medicine 358, 667-675.
Zoghbi, H. Y., and Bear, M. F. (2012). Synaptic dysfunction in neurodevelopmental disorders associated with autism and intellectual disabilities. Cold Spring Harbor perspectives in biology 4.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating a psychiatric disorder in a subject having a 16p11.2 microdeletion syndrome, comprising the step of administering a composition that includes a Group I mGluR inhibitor.

2. The method of claim 1, wherein the psychiatric disorder is schizophrenia.

3. The method of claim 1, wherein the psychiatric disorder is a neuropsychiatric disorder.

4. The method of claim 1, wherein the neuropsychiatric disorder is at least one member selected from the group consisting of anxiety and attention deficit hyperactivity disorder.

5. The method of claim 1, wherein the Group I mGluR inhibitor is a Group I mGluR negative allosteric modulator.

6. The method of claim 5, wherein the Group I mGluR negative allosteric modulator includes an mGluR5 negative allosteric modulator.

7. The method of claim 5, wherein the mGluR5 negative allosteric modular includes a compound comprising:

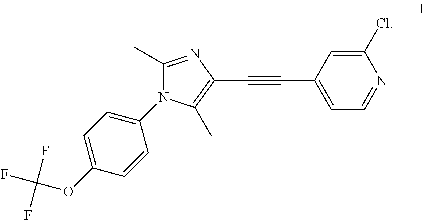 [SMILES: Cc1nc(C#Cc2ccnc(Cl)c2)c(C)n1-c1ccc(OC(F)(F)F)cc1]

8. The method of claim 1, wherein the subject administered the Group I mGluR inhibitor further has autism spectrum disorder.

9. The method of claim 1, wherein the subject administered the Group I mGluR inhibitor further has an improvement in a cognitive impairment following administration of the Group I mGluR inhibitor.

10. The method of claim 9, wherein the improvement in the cognitive impairment is an improvement in at least one member selected from the group consisting of memory, attention and executive function.

11. The method of claim 1, wherein the Group I mGluR inhibitor includes an mGluR1 inhibitor.

12. The method of claim 1, further including the step of administering an mGluR7 inhibitor to the subject.

13. The method of claim 1, wherein the Group I mGluR inhibitor include an mGluR5 inhibitor.

14. The method of claim 1, wherein the subject administered the Group I mGluR inhibitor further has at least one additional condition selected from the group consisting of obesity, an intellectual disability and a seizure disorder.

15. A method of treating a psychiatric disorder in a subject having a 16p11.2 microdeletion syndrome, comprising the step of administering a composition that includes a Group I mGluR negative allosteric modulator.

16. A method of treating a psychiatric disorder in a subject having a 16p11.2 microdeletion syndrome, comprising the step of administering a composition that includes a Group I mGluR antagonist.
